(19) United States
(12) Patent Application Publication
(10) Pub. No.: US 2006/0040390 A1
(43) Pub. Date: Feb. 23, 2006

(54) Device, method, system, and program for intelligent in vivo cell-level chemical or genetic material delivery

(76) Inventors: John Scott Minor JR., Cleveland, OH (US); Laura Boyd Minor, Cleveland, OH (US); John Scott Minor III, Cleveland, OH (US)

Correspondence Address: John Scott Minor, Jr., 1599 Maywood Road, Cleveland, OH, 44121, US

(21) Appl. No.: 10/922,477

(22) Filed: Aug. 19, 2004

(51) Int. Cl. C12N 15/87; A61M 31/00

(52) U.S. Cl. 435/455; 604/500

(57) ABSTRACT

The invention provides a device, method, system and program for intelligent in vivo cell-level chemical or genetic material delivery; wherein multiple injectable biocompatible physical delivery device containers are used to selectively administer medicine, chemical(s) or genetic materials to a target cell in a patient, human or animal, with reduced systemic toxicity; said delivery device container includes an internal contents-to-cell transfer mechanism, usually a syringe; a biological “key” molecule, magnetic device or vibration frequency signature sensor placed on the surface of the delivery device container which is adapted to selectively bind to said target cell directly or indirectly; a “tag” placed on the surface of the delivery device container, usually metallic and biocompatible in nature, which will display to an observer when scanned through external devices such as x-ray, MRI, CT, sound, etc.; and a release mechanism to move the internal contents of the delivery device container into said target cell over a predetermined and specified timed basis.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, TABLE, OR COMPUTER PROGRAM

Not Applicable

### BACKGROUND OF THE INVENTION

1. Field of the Invention

Currently, many cell-level medicines and genetic and chemical therapies for both attack and defense are administered either orally or by injection and work systemically—even though they are to target only specific problem regions or cells within the body.

This systemic based approach is not optimal in its intended efficacy and also does great peripheral damage to non-intended areas of the cells or body—human or animal—which produces many side-effects that vary from mild to possible death.

Introducing therapeutic agents can occur via a direct needle based injection or by a trans-dermal patch, which is a set of needles that allow therapeutic agents to enter the body without the needle penetrating to a depth of skin that will reach nerves. There has been a great deal of difficulty in building a functional transdermal medicinal patch. Current technology can build the micro-needles used to transfer the therapeutic agent into the body, but the micro-needles are very brittle and easily broken—reducing the effectiveness of the approach and adding production costs. While we will demonstrate and expand our invention later in this document as the solution to the problems presented with the prior art for in vivo delivery of therapeutic agents directly to target cells, the same primary embodiment delivery device container construction material, amorphous metal, used for that application will be shown to fix the brittle needle problem for administering our invention.

Micro-technology (objects with dimensions in the 1×10 to the −6 power of a meter) and nano-technology (1×10 to the −9 power of a meter) manufacturing processes and procedures have allowed great strides to be made in both products and materials for many applications.

One such material that can be individually designed for specific chemical, structural and electronic material properties is amorphous metal—which, as the name implies, has the same internal crystalline structure as glass. Because the material properties are designed around this basic crystalline structure, various percentages of different ingredients can be mixed to optimize for specific material properties. Therefore, each therapeutic application can have a unique set of material properties to allow optimization of the total process.

In view of the foregoing disadvantages inherent in the known types of systems now present in the prior art, the present invention provides an improved device, method, system, and program for intelligent in vivo cell-level chemical or genetic material delivery. As such, the general purpose of the present invention, which will be described subsequently in greater detail, is to provide a device, method, system, and program for intelligent in vivo cell-level therapeutic, chemical or genetic material delivery which has all the advantages of the prior art mentioned heretofore and many novel features that result in a device, method, system, and program which is not anticipated, rendered obvious, suggested, or even implied by the prior art, either alone or in any combination thereof. The device, method, system, and program have particular utility in broadly and economically deploying safe target-cell-specific therapies with optimized results given any injection site. The invention solves the above set of problems efficiently and cost effectively. It does this through the use of very small (micro or nano depending on dosage and therapy process) delivery device containers that contain the necessary therapeutic agent(s) and a target cell biological “key” molecule(s) (monoclonal antibody as an example), magnetic device, or vibration frequency signature sensor to provide attachment to only specific target cells. Previous to this invention, the above set of circumstances presented an arduous, costly, painful and possibly life threatening set of tasks, which can now be automated with direct injectable containers at any body site.

This population of humans and animals with unintended systemic side-effects—who will be direct beneficiaries of my invention—from various systemic based therapies (chemotherapy as an example) requires a drug/chemical/gene capable delivery device that targets only the specific sites or cells within the body that are to be treated.

Once the above need is established—which we further address in the following paragraphs, the question becomes what is not anticipated, rendered obvious, suggested, or even implied by the prior art, either alone or in any combination thereof, for an invention that will address these primary medical, genetic or chemical efficacy and, larger issue, un-intended side-effect, problems.

2. Description of the Prior Art

2.a. Heat Based Approaches

A therapeutic procedure explored in some fields of surgery is to generate heat in vivo at specific locations in the body, and to benefit from the heat for therapeutic purposes, such as the treatment of cancer cells. Local heat may be achieved by several methods, e.g. with catheters equipped with elements generating heat by electrical resistivity, which can be controlled to desired locations via the vascular system. This method requires invasive surgery, and, further, requires that every cancerous cell be pinpointed for destruction. If the cancer has metastasized from the primary site, this approach for finding individual cells becomes very difficult, and healthy cells are often destroyed in the name of trying to get all of the cancer.

An alternative technique to achieve heat in-vivo, is to apply small volumes of slurries or pastes of heat generating materials at the desired locations, e.g. by injection with needles. The material injected into the body cures through exothermal chemical reactions and thereby generates the desired temperatures. As the temperature rises, local therapeutic effects are generated. Ideally, when the reactions are completed, the cured substance should form a biocompatible solid material, which can be left for prolonged periods of time in the body without any negative health effects. Only a few types of therapies benefiting from heat generating materials are performed today; the heat generating material being PMMA (polymethylmethacrylate) bone cement, despite the lack of biocompatibility. Again, this approach does not contain the capability to attack every individual problem cell. The material is applied in a mass and kills a large group of cells without any certainty that all of the problem cells have been found and destroyed.

Treatment of malignant cancerous tumors, as well as metastasis, myeloms, various cysts, etc, involving the local application of heat generating materials in vivo is used to some degree, although it is still a less frequent treatment technique. The technique involves either local thermal necrosis or restriction of the nutritional or blood feed, or oxygenation, to the tumors or cells. The approach requires invasive techniques and does not work well when cancer cells might have moved from the original tumor site.

The use of inject-able heat generating materials for cancer treatment is particularly suitable for tumors in the skeleton. The procedure may involve direct injection of a cell-destroying cement; or alternatively the removal of the tumor by surgery, followed by filling of the remaining cavity by an in-situ-curing material. The former procedure offers at least two advantages: One being that increased temperatures during curing reduce the activity of, or kills, residual cancerous tissue. The second effect is that the cement restores the mechanical properties of the skeleton, hence reducing the risk of fractures due to weakened bone.

Inject-able pastes are also used in combination with radiation treatment, as when spine vertebrae are first filled with PMMA bone cement injected into the trabecular interior through the pedicles to provide mechanical stability, followed by radiation treatment of the same vertebra. As above, our invention does not address mechanical or structural integrity. However, the second step, radiation, is a non-specific approach for killing all cells that it touches.

Similarly, inject-able pastes are used for the treatment of collapsed osteoporotic vertebrae. The filling of collapsed vertebrae with bone cement reduces the pain and the dimensions of the vertebrae may be restored. Here the heat generation contributes, in addition to the mechanical stabilization of the vertebrae, to the reduction of pain in the spine.

Locally generated heat can be used for the local destruction of nerves to reduce pain, to destroy the function of blood vessels, and to locally trigger the effect of drugs. However, the approach works on large groups of cells without differentiation.

Further, PMMA based bone cements are also not biocompatible materials. They have clear toxic side-effects caused by leakage of components, such as solvents and non-polymerized monomer. These leakages become particularly high for low viscosity formulations (being inject-able) with high amounts of solvents and monomers.

Today's bone cements cure while generating heat in amounts considered excessive for normal orthopedic use. For use in vertebroplasty, some argue that a temperature rise may be advantageous, since it may contribute to reduce pain. However, today's bone cements offer no, or very limited, possibilities for the surgeon to control the generated temperature—causing additional side-effects and healthy cell damage.

Also, cements generating low temperature rises during curing are of interest. A low temperature bone cement based on hydraulic ceramics is described in the Swedish patent application “Ceramic material and process for manufacturing” (SE-0104441-1), filed 27 Dec. 2001. In said patent application the temperature rise due to the hydration reactions is damped by addition of suitable inert, non-hydraulic phases, which are also favorable for the mechanical properties and biocompatibility. However, these ceramic materials do not offer the means to control the heat generation through well controlled phase compositions of the hydrating ceramic, or controlling the temperature by accelerators and retarders—which lessens their overall effectiveness in directly addressing all cancer or problem causing cells for the whole body.

2.b. Molecule Linking Based Approaches

Medical Applications

The clinical use of chemotherapeutic agents against malignant tumors is successful in many cases, but also has several limitations. These agents do not affect tumor cell growth selectively over rapidly growing normal cells, leading to high toxicity and side effects. For example, paclitaxel and related taxanes are a very potent class of anticancer drugs first isolated in 1971. Paclitaxel has a unique mechanism of action, it promotes microtubule polymerization leading to abnormally stable and nonfunctional microtubules. Hence, cells are blocked at the G2-M phase of the cell cycle, leading to apoptotic death.

Paclitaxel has clinical efficacy, despite several problems associated with poor solubility and high toxicity. Clinical trials showed remarkable efficacy against advanced solid tumors such as ovarian and breast cancer and a panel of other tumors. Most of the side effects of taxanes occur at rapidly growing tissue such as bone marrow, hematopoietic, and gut epithelia. Because microtubule function is key for neuronal survival, neurotoxicity is also a problem for taxanes.

Doxorubicin is one of the most widely used anticancer agents. It has a strong antiproliferative effect over a large panel of solid tumors. Doxorubicin intercalates into DNA and breaks the strands of double helix by inhibiting topoisomerase II.

Despite its clinical efficacy, Doxorubicin suffers a major drawback which is common of all chemotherapeutic agents: it is not tumor selective and therefore affects healthy tissue as well causing severe side effects, including cardiotoxicity and myelosuppression (Tewek K. M. et al. Science 226: 466468, 1984).

Moreover, the intrinsic or acquired resistance of cancer cells to Doxorubicin is another factor that limits its efficacy. For instance, the multidrug resistance associated p-glycoprotein (p-gp) is a transmembrane pump that facilitates active cellular efflux of toxic compounds and, thereby, lowers cytotoxicity of the drugs (Zhang D. W. et al. J. Biol. Chem. 276(16): 13231-9, 2001). Verapamil is a p-glycoprotein inhibitor commonly used in drug resistance studies.

Several approaches have been developed in order to specifically deliver Doxorubicin to tumor cells using monoclonal antibodies (mAbs) or small peptides as carrier molecules. Site-directed delivery may increase the intratumor concentration of the drug while decreasing systemic toxicity.

The outcome of linkage-based targeted chemotherapy greatly depends on two factors: the ability of the carrier molecule to selectively recognize tumor cells and the nature of the chemical linkage used for coupling the cytotoxic agent to the carrier. Ideally, the conjugate should be stable and inactive in the circulation, with the cytotoxic radical released in its active form in the target tumor tissue after internalization of the conjugate (Guillemard V. et al. Cancer Res. 61:694-699, 2001). Few situations produce “ideal” circumstances.

Different chemical strategies have been used to couple drugs to mAbs including periodate oxidation of mAbs. Using this approach, diols located in the antibody's carbohydrate chains are cleaved by periodate to form reactive aldehyde groups which can further react with amine or hydrazide residues forming a C—N linkage. The main advantage of this technique is that the carbohydrate residues that are affected are usually located at distant sites from the antibody's binding regions. Thus, modification of the antibody through these residues should have little or no effect on the antibody's activity. While somewhat effective, the process lacks the ability to build up medicine, chemical or genetic materials quickly in the target cell—removing the possibility of total eradication or total genetic remediation of the target-cells in a single dose. As well, due to the fact that multiple doses are usually required with the linkage approach, the initial dose may remove the receptors on the surface of the target cell—removing the ability to revisit the target cell for a multiple dose regimen.

The problem of selectivity can be addressed by using monoclonal antibodies (mAbs) as a “key” that target “tumor markers”, which are proteins generally over-expressed on the surface of tumor cells. In passive immunotherapy, mAbs can act either as pharmacological agents, as adjuvants or as cytotoxic agents upon fixation of complement, and as carriers for large toxins or cytokines. However, mAbs are generally poor pharmaceuticals and are poor cytotoxic agents.

Many cancer cell types over-express certain cell surface components such as proteins or glycolipids, which are known as tumor markers. Examples include receptor tyrosine kinases such as type I insulin-like growth factor receptor (IGF-R) and Her2/neu, TrkA, etc. It would be desirable to target higher amounts of chemotherapeutics to these tumor markers to achieve totally selective tumor death with no systemic toxicity.

Another method for reducing toxicity would be to selectively deliver protective agents to non-tumor cells. For example the neurotoxicity caused by the chemotherapeutic agent taxol may be ameliorated by selective delivery of neuroprotective agents to neurons in a manner that does not alter the desired tumor killing of non-neuronal cells.

International application published under No. WO 00/33888 discloses a modified form of a therapeutic agent which comprises a therapeutic agent, an oligdpeptide, a stabilizing group and, optionally, a linker group.

U.S. Pat. Nos. 4,997,913 and 5,084,560 disclose a pH-sensitive immunoconjugates which dissociate in low-pH tumor tissue, comprising a chemotherapeutic agent and an antibody reactive with a tumor-associated antigen. pH sensitive immunoconjugates are effective in specific pH situations only, while also allowing the chemotherapeutic agent to be deployed in non-tumor regions.

International application published under No. WO 96/09071A1 discloses a conjugate consisting of an active substance and a native protein which is not considered exogenous. The conjugate is distinguished in that, between the active substance and the protein, there is a linker which can be cleaved in a cell. The linker based approach provides for a lower amount of chemotherapeutic agent to be administered to the target tumor cell than with our invention—reducing the kill rate—because the active substance must remain inactive as it travels through the body. The chemistry of this approach also limits the makeup of the active substance.

U.S. Pat. No. 6,030,997 discloses a pharmaceutically acceptable pro-drug which is a covalent conjugate of a pharmacologically active compound and a blocking group, characterized by the presence of a covalent bond which is cleaved at pH values below 7.0. pH based approaches limit the range of cell types that can be addressed.

U.S. Pat. No. 6,140,100 discloses a conjugate of a cell targeting molecule and a mutant human caboxypeptidase A enzyme. Conjugate approaches do not contain the volume of material to a single target tumor cell needed for killing the cell on a single application.

U.S. Pat. No. 5,208,323 discloses an anti-tumor compound which comprises an antibody used to target the anti-tumor agent to the malignant cells. These approaches, while effective, can not provide the higher degree of administered material that is required to kill the cell on the first application.

2.c. Encapsulation Based Approaches

Medical Applications

U.S. Patent Application No. 20020155144 discloses a biofunctional hydroxyapatite coating and microsphere for in-situ drug encapsulation. The invention relates to a room-temperature process for obtaining calcium phosphate, in particular, hydroxyapatite, coatings and microspheres that encapsulate drugs for subsequent controlled release. This process addresses time release issues only and is not target-cell specific.

Problems with drug delivery in vivo are related to toxicity of the carrier agent, the generally low loading capacity for drugs as well as the aim to control drug delivery resulting in self-regulated, timed release. With the exception of colloidal carrier systems, which support relatively high loading capacity for drugs, most systems deliver inadequate levels of bioactive drugs. In terms of gene delivery, to date, the most efficient, though least safe, methods of delivery are through viral mediated gene transfer. It is a highly inefficient method, and is faced with even greater problems than the delivery of drugs due to the hydrophilic and labile nature DNA oligos. The problems with delivery of genes or antisense oligos originate from the rapid clearance of plasmid DNA or oligos by hepatic and renal uptake as well as the degradation of DNA by serum nucleases [Takura Y, et al. Eur. J. Pharm. Sci. 13 (2001) 71-76]. These effects have been observed for both in-situ and intravenous delivery. For example it was estimated that more than half of the intravenous or in-situ delivered naked plasmid DNA was cleared from the tumor site within the first two hours following intratumoral injection [Ohkouchi, K., et al Cancer Res. 50, (1996) 1640-1644 and Imoto, H., et al. Cancer Res. 52, (1992), 4396-4401]. After the clearance loss, only a small percentage of the remaining DNA or oligos make their way to the cytoplasm or nucleus of the target cell. The membrane permeability of naked DNA and especially oligos is virtually nonexistent, due to their polyanionic nature. For this reason, their uptake through the endosomal compartment is associated with a severe drop in pH and degradation. Finally, many of the genes delivered have to be transported and sometimes incorporated in the genome of the target cell for stable expression. This makes very difficult gene transfer in vivo. In addition, successful controlled release is still problematic as for most applications (with the exception of naked DNA vaccines) it is desirable to have a prolonged expression of the gene of interest to ameliorate a particular medical condition. In most applications anywhere from a few weeks to several months are desired for the expression of a certain gene product.

Drug encapsulation in HA has been achieved in the past by simple post-impregnation of a sintered, porous HA ceramic [K. Yamamura et al, J. Biomed. Mater. Res., 26, 1053-64, 1992]. In this process, the drug molecules simply adsorb onto the surface of the porous ceramic. The drug release is accomplished through desorption and leaching of the drug to the surrounding tissue after exposure to physiological fluid. Unfortunately, most of the adsorbed drug molecules release from such a system in a relatively short period of time. Impregnation of drug material into porous sintered calcium phosphate microspheres has been reported in patent literature. “Slow release” porous granules are claimed in U.S. Pat. No. 5,055,307 [S. Tsuru et al, 1991], wherein the granule is sintered at 200-1400 C and the drug component impregnated into its porosity. “Calcium phosphate microcarriers and microspheres” are claimed in WO 98/43558 by B. Starling et al [1998], wherein hollow microspheres are sintered and impregnated with drugs for slow release. D. Lee et al claim poorly crystalline apatite [WO98/16209] wherein macro-shapes harden and may simultaneously encapsulate drug material for slow release. It has been suggested to use porous, composite HA as a carrier for gentamicin sulfate (GS), an aminoglycoside antibiotic to treat bacterial infections at infected osseous sites [J. M. Rogers-Foy et al, J. Inv. Surgery 12 (1997) 263-275]. The presence of proteins in HA coatings did not affect the dissolution properties of either calcium or phosphorus ions—it was solely dependent on the media [Bender S. A. et al. Biomaterials 21 (2000) 299-305].

A group at Kobe University lead by Prof. M. Otsuka performed a series of investigations of drug encapsulation in self-setting calcium phosphate cements derived from tetracalcium phosphate and dicalcium phosphate [J. Contr. Rel. 43 115-122 1997; ibid 52 281-289 1998; J. Pharm. Sci. 83 611-615, 259-263, 255-258, 1994]. The cement was shaped with an in-situ drug encapsulation, into 15 mm diameter macro-pellets and drug (indomethacin) release monitored over a 3 week period. It was concluded that the cement-drug delivery system, shaped in-situ into surrounding bone tissue, may be an excellent way to treat localized bone infections with high therapeutic effectiveness. The advantage of HA for drug delivery is that side effects have never been a concern for hydroxyapatite materials [Y. Shinto et al, J. Bone Jt. Surg., 74B4, 600-4, 1992]. Calcium phosphate—biodegradable polymer blends were also investigated as possible vehicles for drug delivery [I. Soriano and C. Evora, J. Contr. Rel. 68 121-134 2000]. Prolonged drug release (up to 10 weeks) was obtained for the composites coated with hydrophobic polymer coatings. A group from the University of Pennsylvania [Q. Qiu et al J. Biomed Mat Res. 52 66-76 2000] processed polymer-bioactive glass-ceramic composite microspheres for drug delivery. Porous calcium phosphate ceramics were impregnated with bone marrow cells [E. Kon et al, J. Biomed Mat. Res. 49 328-337 2000] and with human bone morphogenetic protein [I. Alam et al J. Biomed Mat. Res. 52 2000]. The above is an in-situ approach.

S. Takenori et al., in U.S. Pat. No. 5,993,535 (and accompanying EP0899247), disclosed a calcium phosphate cement comprising tetracalcium phosphate and calcium hydrogen phosphate polysaccharide as main components. It needed 24 hours incubation at 37.degree C. for conversion of hydroxyapatite. T. Sumiaki et al., in U.S. Pat. No. 5,055,307, disclosed slow release drug delivery granules comprising porous granules of a calcium phosphate compound having a ratio of Ca to P of 1.3 to 1.8, a porosity of 0.1 to 70%, a specific surface area of 0.1 to 50 m.sup.2/g and a pore size of 1 nm to 10 .mu.m. The granules were fired at a temperature of 200 to 1400.degree. C., and a drug component impregnated in pores of the granules, and a process for producing the same. S. Gogolewski, in WO00/23123, disclosed the hardenable ceramic hydraulic cement comprising a drug to be delivered to the human or animal body upon degradation or dissolution of the hardened cement. However, conversion of CPC to achieve HA needed 40 hours. L. Chow et al., in U.S. Pat. No. 5,525,148, disclosed calcium phosphate cements, which self-harden substantially to hydroxyapatite at ambient temperature when in contact with an aqueous medium. More specifically the cements comprise a combination of calcium phosphates other than tetracalcium phosphate with an aqueous solution adjusted with a base to maintain a pH of about 12.5 or above and having sufficient dissolved phosphate salt to yield a solution mixture with phosphate concentration equal to or greater than about 0.2 mol/L. However, disadvantages of the process are that high pH (>12.5) could denature most drugs, proteins and DNA, so the process is not suitable for drug encapsulation vehicles. C. Rey et al., in WO9816209, disclosed a synthetic, poorly crystalline apatite calcium phosphate containing a biologically active agent and/or cells, preferably tissue-forming or tissue-degrading cells, useful for a variety of in vivo and in vitro applications, including drug delivery, tissue growth, and osseous augmentation. However, the ratio of Ca/P was limited to less than 1.5, and the authors did not disclose how to fabricate the microspheres and coatings.

U.S. Patent Application No. 20030211165 discloses an injectable composition comprising biocompatible, swellable, substantially hydrophilic, non-toxic and substantially spherical polymeric material carriers which are capable of efficiently delivering bioactive therapeutic factor(s) for use in embolization drug therapy. It further relates to methods of embolization gene therapy, particularly for the treatment of angiogenic and non-angiogenic-dependent diseases, using the injectable compositions. This approach requires that the spheres be injected directly to the embolization site.

2.d. Magnetic Device Based Approaches

Medical Applications

Researchers Zachary Forbes and Benjamin Yellen, biomedical engineering doctoral students at Drexel University, Philadelphia, Pa., have developed a weak magnetic alloy stent device with an externally controllable magnetic field. Medications are then introduced into the body in biospheres that are metallic and thus respond to the pull of the stent's magnetic field. While effective for replenishing stent based therapeutic (usually for scar tissue prevention in cardiac artery obstructions) agents over the life of the patient, the approach requires an implanted magnetic metal object which can be turned on or off via the external magnetic field—thus it provides no means for addressing individual cells.

2.e. Cell Vibration Signature Based Approaches

Medical Applications

Researcher and nano-scientist Jim Gimzewski, University of California, Los Angeles, Los Angeles, Calif., has developed a nano-acoustical sensor that allows the natural vibration of cells to be observed. Currently, the sensor must be associated to an individual cell via an invasive procedure. Research will continue in order to provide the sensor device in a form that can be administered via injection. Healthy cells (a yeast cell, for example, displays about 1,000 vibrations per second) are expected to have vibration signatures different from unhealthy cells, so this device may allow cell differentiation in a totally different approach than the bioreceptors now used for discerning target cells.

### BRIEF SUMMARY OF THE INVENTION

In view of the foregoing disadvantages inherent in the known types of systems now present in the prior art, the present invention provides an improved device, method, system, and program for intelligent in vivo cell-level chemical or genetic material delivery. As such, the general purpose of the present invention, which will be described subsequently in greater detail, is to provide a device, method, system, and program for intelligent in vivo cell-level therapeutic, chemical or genetic material delivery which has all the advantages of the prior art mentioned heretofore and many novel features that result in a device, method, system, and program which is not anticipated, rendered obvious, suggested, or even implied by the prior art, either alone or in any combination thereof. The device, method, system, and program have particular utility in broadly and economically deploying safe target-cell-specific therapies with optimized results given any injection site. The invention solves the above set of problems efficiently and cost effectively. It does this through the use of very small (micro or nano depending on dosage and therapy process) delivery device containers that contain the necessary therapeutic agent(s) and a target cell biological “key” molecule(s) (monoclonal antibody as an example), magnetic device, or vibration frequency signature sensor to provide attachment to only specific target cells. Therapeutic agent injection is then provided on a predetermined and specified timed basis. Previous to this invention, the above set of circumstances presented an arduous, costly, painful and possibly life threatening set of tasks, which can now be automated with direct injection-able containers at any body site.

A review of the invention design material properties is provided as follows. The first material property is the ability to be benign with regard to bodily defenses. The body should not see the device as foreign or as a target for attack. In our design this is accomplished with amorphous metal by including titanium in the material chemical mixture.

The second material property addresses the chemical transfer from the carrying device to the target cell which must be done in such a way that the chemical is transferred in its majority directly into the targeted cell—minimizing adjacent cell un-intended damage. The two primary elements of our design that are addressed with this material property are the container design and the container-to-target-cell transfer mechanism (a syringe as an example). Amorphous metal has the ability to be sharp when first manufactured. There is no need for the additional time and manufacturing cost of secondary procedures. Therefore, the syringe needle and container-to-target-cell transfer mechanism developed for cell penetration with this invention can be built within the same manufacturing process as the building of the container itself. Amorphous metal is also very strong, and the container design provided here (a sphere in the primary embodiment) has the added structural ability to contain an internal pressure that is much higher than that internal to the target cell.

The third material property addresses the biological “key” molecule (an example is a monoclonal antibody), a magnetic device or a vibration frequency signature sensor that must be able to be attached to the delivery device container so that this “key” will find and attach to only the specified target cell's corresponding “latch”. The use of antigens, which have a corresponding receptor on the target cell, is well known in the industry, and is a standard approach for individual cell specificity requirements. Amorphous metal has many chemical combinations, and the binding molecule can be attached in either a mechanical or a chemical approach to reflect the requirements of specific binding molecules.

The forth material property is that the device be able to be manufactured easily and cost effectively and in such a way that it will act as a secure container for the directed chemical. Multiple manufacturing approaches are now available through the MEMS (microelectromechanical systems) and nano industries, which range from etching technology to spray “ink” technology to laser cutting technology. All of these approaches allow a secure container system to be built in volume, and amorphous metal, which is strong, durable, and has many chemical combinations, provides an easy material with which to work at these very small sizes.

The fifth material property is that the delivery device also have a “tag” that would be displayable on some form of external scanning device. Examples of the external scanning device would be x-ray, sound, magnetic resonance, etc. A common “tag” is a gold nano-particle which is benign when used inside the body but has the value of showing on various external scanning devices. The amorphous metal device, depending on the chemical makeup, may also be obvious in the scanning process which may remove the need for additional “tag” materials. This “tag” process allows for verification that the device has found its intended bodily region or specified cell while also giving some indication of target cell volumes and any unknown or unexpected locations.

The sixth material property is that a medicine, chemical or genetic material delivery device container-to-target-cell transfer device be able to be built at micro and nano sizes. Amorphous metal, as one such material possibility, can be used to produce micro or nano scale devices. Should a syringe design, as in embodiment 1, be used for the container-to-target-cell transfer, amorphous metal also has the ability to be sharp after the first manufacturing step—no secondary sharpening steps are required, so the final true dimensions can be designed and built in the initial stage of manufacture. Pressure based syringe deployment and predetermined and specified time release control mechanisms can also be built on a micro or nano scale using the same build process, allowing the delivery device container to move through the body with no sharp projections until the actual target cell has been reached and the device properly attached.

It would be highly desirable to be provided with a compound as a therapeutical agent to target and kill or protect specific cells in a patient with reduced systemic toxicity.

Furthermore, it would be highly desirable to be provided with mAbs and chemotherapeutics in larger doses or mAbs and protective agents in larger doses, for example, to allow for the delivery of a cytotoxic agent to tumor cells with higher therapeutic capability and reduced systemic toxicity.

Furthermore, it would be desirable to be provided with the ability to change the size of the delivery device container to correspond with any specific requirements of chemotherapeutics or protective agents in any manner.

Furthermore, it would be desirable for an individual container that protects non-target cells from the systemic impact of the above drugs by using mAbs both as a targeting means for specific target-cell binding and as a trigger mechanism for the automated deployment of the container-to-cell device. Further, our invention carries far more of the medicine, chemical or genetic material intended for the target cell—substantially increasing the intended end result of the therapy agent while removing the concerns of unintended systemic side-effect.

Our invention does not use an external chemically active coating that will degrade over some timed period to release its contents. Rather, our invention provides a benign (not seen as invasive by the body's defensive mechanisms) delivery device container that produces no interaction with the therapeutic agent of choice, uses a target-cell specific mAbs for a specified attachment force, and upon said attachment, said force provides the trigger for the mechanical release of the container-to-cell mechanism for moving the contents of the container into the target cell on a predetermined and specified time basis.

Further, our invention has the ability to use a controlled delivery device container-to-cell therapeutic agent release mechanism and a container of any appropriate size which allows for higher initial doses and extended delivery of the therapeutic agent without regard to systemic un-intended side-effects. This produces the earliest desired and most optimized therapeutic effect.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved device, method, system, and program for intelligent in vivo cell-level chemical or genetic material delivery that has all of the advantages of the prior art systems and none of the disadvantages.

The objective of the present invention is to provide injection-able devices that then target individually defined cell(s) systemically (in any body location) for therapeutic treatment by the use of a biological “key” molecule (mAbs as an example), magnetic device, or vibration signature or any other desired targeting method—without regard to the injection site or location of the target cell(s) within the body. Every metastasized target cell will then be found, attached to, and the delivery device container contents delivered over the predetermined and specific timed basis doses required for optimized results.

A further objective of the present invention is to provide injection-able devices that also “tag” individual defined target cell(s) systemically for therapeutic treatment—without regard to the injection site or location of the target cell within the body. This “tag” shall be of a nature that external (non-invasive) analysis or scanning can detect it.

A further objective of the present invention is to provide injection-able devices that also administer on a predetermined and specified timed period or schedule, via container-to-cell transfer, medicines, chemicals or genetic materials directly to an individual target cell systemically for total organism therapeutic treatment—without regard to the injection site or location of the target cells within the body.

More particularly, the injection-able device will be of a material that is biocompatible with the body, human or animal, so that it can be used suitably for therapeutic purposes in vivo in the body without fear of bodily defenses.

The present invention further pertains to a method for manufacturing the above-described container device. One embodiment of a suitable container material is an amorphous metal composition. An amorphous metal is a metallic material (usually an alloy rather than a pure metal) that is noncrystalline; that is, there is no long-range order of the positions of the atoms. Amorphous metals are commonly referred to as metallic glasses which differ from traditional metals in that they have a non-crystalline structure and possess unique physical, electrical and magnetic properties that combine strength and hardness with flexibility and toughness. Amorphous metal based structures can be manufactured via any of the standard micro and nano methodologies. The material can also be sprayed on a sub structure or made into a foamed metal to increase structural integrity without an increase in weight. Though the preferred embodiment is spherical and of amorphous metal, this is not a limiting factor, and any suitable material may be used in other embodiments (another embodiment example being carbon nanotubes).

The present invention also pertains to a therapeutic method comprising the steps of introducing a therapeutic agent over a defined interval on a predetermined and specified timed basis from the container into a target cell in a living body.

An even further object of the present invention is to provide a new and improved device, method, system, and program for intelligent in vivo cell-level chemical or genetic material delivery that has a low cost of manufacture with regard to both materials and labor, and which accordingly is then susceptible of low prices of sale to the consuming public, thereby making such system economically available to the buying public.

The present invention also includes the use of amorphous metal as the material for manufacturing transdermal or transcutaneous micro-needles for therapeutic material delivery via a transdermal or transcutaneous patch. The patch approach also provides a different embodiment of administering the delivery device container.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and detailed descriptive matter in which there are illustrated preferred embodiments of the invention.

### BRIEF DESCRIPTION OF THE DRAWINGS

The present invention will become more fully understood from the detailed description given below and the accompanying drawings which are given by way of illustration only, and thus are not limitative of the present invention, and wherein:

FIG. 1 displays an isometric cut-away of the delivery device container 18. The spring needle deployment assembly 28 is shown in the cocked or non-deployed position—which means the release mechanism is compressed. The mAbs material 16 attached to the fill and needle deployment orifice 10, the pivot mechanism pins 12 as part of the pinned needle deployment force assembly 14 are also shown.

FIG. 2 displays an elevation view of the needle assembly showing the mAbs material 22 attached to the fill and needle deployment orifice (orifice not shown—please see FIG. 1-10), the needle 24 in the deployed position (which would insert it into a target cell), and the needle spring 26 which is shown in the triggered or expanded position.

FIG. 3 shows one example of a manufacturing process for micro or nano sized devices (deposition or “printing” 32) such as the delivery device container 18. Much of the manufacturing tooling and process improvement for these sized objects comes from the printed circuit fabrication industry, so there are multiple processes available from which to choose and all are well known within the micro and nano technology industry.

FIG. 4 shows one example of a therapeutic agent filling process 14 for the delivery device containers 18. The therapeutic agent is injected into the delivery device container 18 through the fill and needle deployment orifice 10. A secondary step in this example is the attachment of the mAbs 22 biological “key” molecule. The medical industry in general has multiple technologies for this application.

FIG. 5 shows an injection needle 16 penetrating the skin for the injection of multiple delivery device containers 18. The injection needle 16 can be from a syringe based approach or from a transdermal patch based approach as examples of delivery device container 18 administration. Once administered, the delivery device container 18 utilizes the mAbs biological “key” molecule 22 to seek out and bind to the target cell 30.

FIG. 6 shows a target cell 30 which in this example is a tumor cell displaying multiple receptors 20 on its surface to which the mAbs biological “key” molecules 22 will bind.

FIG. 7 is a magnification of the initial binding process—showing the target cell 30, the delivery device container 18, the binding of the receptor 20 and the mAbs 22 biological “key” molecule. Not displayed are embodiments that use magnetic or vibration signature based approaches for cell differentiation.

FIG. 8 shows the triggering of the spring needle deployment mechanism 28 as the force of the binding process (target cell 30 receptor 20 with the mAbs 22 biological “key” molecule) allows the expansion of the needle spring 26 which uses the pivot mechanism 12 to push out the therapeutic agent via the pinned needle deployment force assembly 14. The needle 24 then penetrates the target cell 30 and releases the therapeutic agent into the interior of the target cell 30.

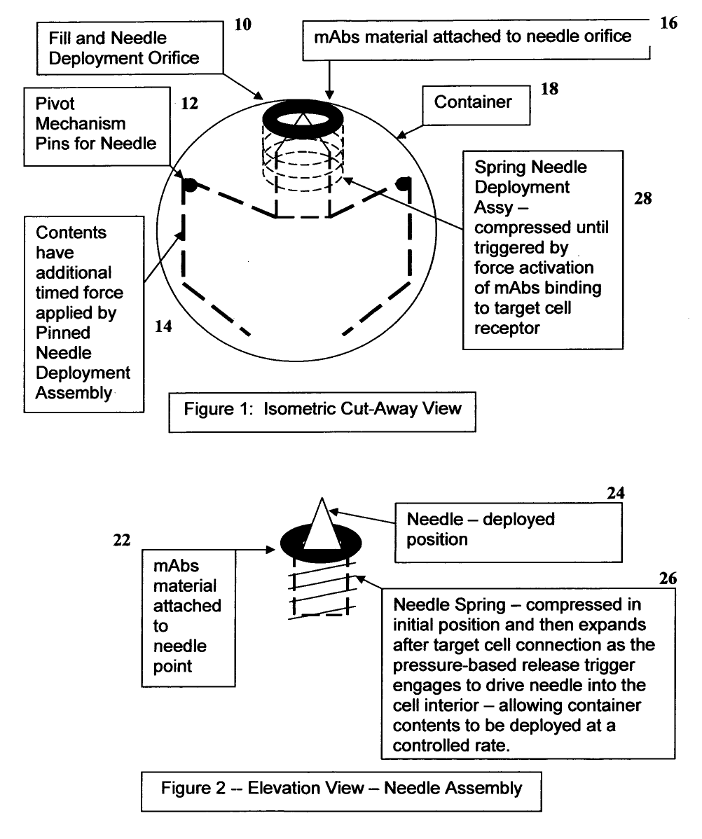

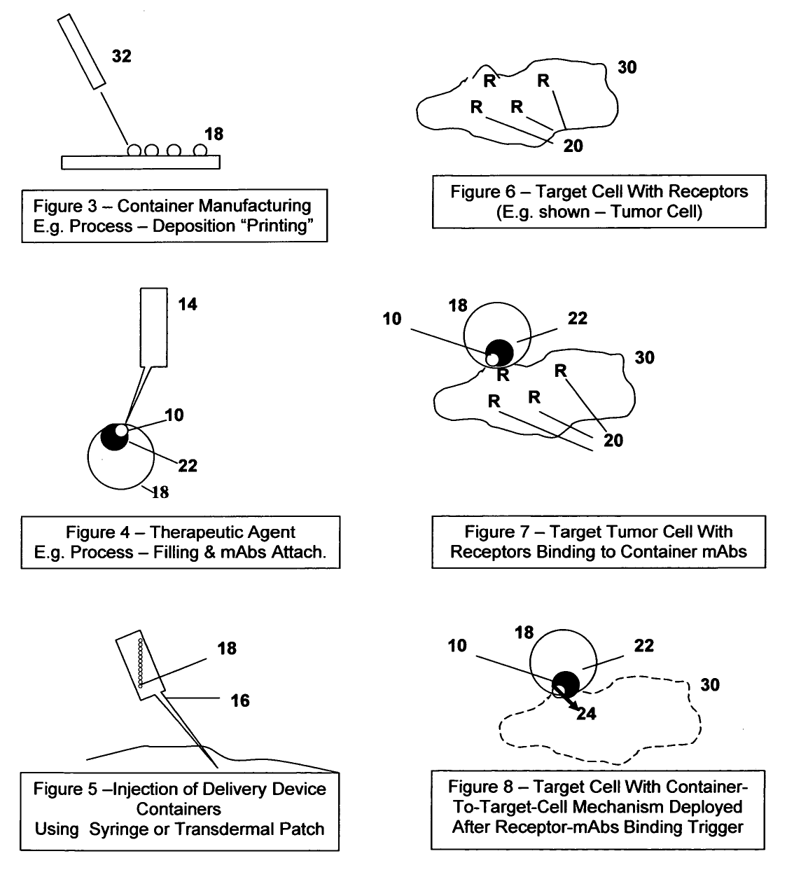

Once the target cell has received the therapeutic agent, the delivery device container 18 can be designed to be biodegradable or to simply be passed through the body as waste.

We have developed various designs for the pinned needle deployment force assembly 14 pressure mechanism which are not shown in order to provide simplicity in the drawing construction. These designs of this pressure mechanism will individually dictate the rate on the predetermined and specified time basis at which the internal contents of the delivery device container will move from the delivery device container into the cell interior.

### DETAILED DESCRIPTION OF THE INVENTION

In the Background section of this document, we related the issues and problems with the current therapeutic systems as well as the larger or superset of issues related to the lack of an economical highly target-cell-specific, open systemic injection site device, method, system, and program for intelligent in vivo cell-level chemical or genetic material delivery. Examples within these areas were reviewed. We then related specific needs and the current prior art with their shortcomings in these areas. Finally, in this section, we will review our invention which addresses the novel, useful and non-obvious device, method, system, and program for intelligent in vivo cell-level chemical or genetic material delivery that fills these needs.

While a preferred embodiment of the device, method, system, and program for intelligent in vivo cell-level chemical or genetic material delivery will be described in detail, it should be apparent that modifications and variations thereto are possible, all of which fall within the true spirit and scope of the invention. With respect to the detailed description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the detail provided is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

A therapeutic agent can be used to kill a cell or to safeguard a cell. Examples of a therapeutic agent can be selected from the group consisting of chemotherapeutic agent, antiviral agent, antibacterial agent, antifungal agent and enzyme inhibitor agent.

A biological “key” molecule, magnetic device, or vibration signature is adapted to selectively bind the delivery device container to the target cell directly or indirectly.

The therapeutic agent, in accordance with a preferred embodiment of the present invention, when the delivery device container is bound to the target cell, is internalized into the target cell via a container-to-cell mechanism, an example being a syringe.

The therapeutic agent, in accordance with a preferred embodiment of the present invention, wherein the chemotherapeutic agent is selected from the group of taxanes, taxanes derivatives, anthracyclines, anthracyclines derivatives, doxorubicin, daunomycin, daunorubicin, adriamycin, methotrexate, mitomycin, epirubicin, nucleoside analogs, DNA damaging agents and tyrphostins.

The therapeutic agent, in accordance with a preferred embodiment of the present invention, wherein the protective agent is an enzyme inhibitor such as a caspase inhibitor agent.

The therapeutic agent, in accordance with a preferred embodiment of the present invention, wherein the therapeutic agent is antisense oligonucleotide or a cDNA for a gene.

The therapeutic agent, in accordance with a preferred embodiment of the present invention, wherein the taxane is paclitaxel.

The therapeutic agent, in accordance with a preferred embodiment of the present invention, wherein the chemotherapeutic agent is doxorubicin.

The therapeutic agent, in accordance with a preferred embodiment of the present invention, wherein the biological “key” molecule is selected from the group of antibody and mimicking molecules thereof, peptides, peptidomimetics, growth factors, hormones, adhesion molecules, viral proteins and functional fragments thereof.

The biological “key” molecule, in accordance with a preferred embodiment of the present invention, wherein the antibody is a monoclonal antibody.

The biological “key” molecule in accordance with a preferred embodiment of the present invention, wherein the antibody binds to a specific receptor on the target cell.

The biological “key” molecule, in accordance with a preferred embodiment of the present invention wherein the monoclonal antibody is MC192 (p75 binding), or 5C3 (TrkA binding), or a-IR3 (IGF-1 R binding).

The biological “key” molecule, in accordance with a preferred embodiment of the present invention, wherein when a primary biologically active molecule indirectly binds to the target cell, the biological “key” molecule further comprises a second molecule which is a secondary biologically active molecule selectively bound to the first and adapted to selectively bind to the target cell.

The biological “key” molecule, in accordance with a preferred embodiment of the present invention, wherein the primary and/or the secondary biologically active molecule is an antibody.

The biological “key” molecule, in accordance with a preferred embodiment of the present invention, wherein a primary antibody is of a species and a secondary antibody is of a different species.

The biological “key” molecule, in accordance with a preferred embodiment of the present invention, wherein the secondary biologically active molecule is a rabbit-antimouse antibody.

In accordance with the present invention, there is provided in the delivery device container a therapeutic agent, which comprises a therapeutically effective amount of the therapeutic agent in association with a pharmaceutically acceptable carrier.

In accordance with the present invention, there is provided an anti-cancer therapeutic agent, which comprises a therapeutically effective amount of a compound of a chemotherapeutic agent in association with a pharmaceutically acceptable carrier.

In accordance with the present invention, there is provided a method for treating cancer with reduced effects in a patient, the method consisting in administering a therapeutically effective amount of a chemotherapeutic agent in association with a pharmaceutically acceptable carrier to target cells in a patient.

In accordance with the present invention, there is provided a method for decreasing toxic side effects and increasing selectivity of a chemotherapeutic agent for tumor cells, the method comprising the step of: administering to a patient a device, method, system and program for intelligent in vivo cell-level chemical or genetic material delivery; wherein multiple injectable biocompatible physical delivery device containers are used to selectively administer medicine, chemical(s) or genetic materials to target cells in a patient, human or animal, with reduced systemic toxicity; said delivery device container includes an internal contents-to-cell transfer mechanism, usually a syringe; a biological “key” molecule placed on the fill and needle deployment orifice of the delivery device container which is adapted to selectively bind to said target cell directly or indirectly; a “tag” placed on the surface of the delivery device container, usually metallic and biocompatible in nature, which will display to an observer when scanned through external devices such as x-ray, MRI, CT, sound, etc.; and a release mechanism to move the internal contents of the delivery device container into said target cell over a predetermined and specified timed basis.

In accordance with the present invention, there is provided a method for by-passing resistance of tumor cells by p-glycoprotein pump (PGP), the method comprising the step of administering the compound of the present invention to a patient in need of such a treatment whereby the biologically active “key” molecule is a monoclonal antibody and the therapeutic agent avoids the membrane diffusion/permeability route to enter into the cells directly via the container-to-cell transfer mechanism, usually a syringe.

In accordance with the present invention, there is provided a therapeutic agent to selectively protect a target cell which comprises a therapeutic agent from the group consisting of: enzyme inhibitors such as caspase inhibitors, ligands of nuclear receptors, vitamin D, vitamin E and their analogs, estrogen and its analogs and inhibitors of the apoptotic cascade using a biologically active “key” molecule which is adapted to selectively bind to the target cell directly or indirectly.

In accordance with the present invention, there is provided a method for decreasing toxic side effects to non-tumor cells, the method comprising the step of administering to a patient a device, method, system and program for intelligent in vivo cell-level chemical or genetic material delivery; wherein multiple injectable biocompatible physical delivery device containers are used to selectively administer protective medicine, chemical(s) or genetic materials to target cells in a patient, human or animal, with reduced systemic toxicity; said delivery device container includes an internal contents-to-cell transfer mechanism, usually a syringe; a biological “key” molecule(s) placed on the surface of the delivery device container which is adapted to selectively bind to said target cell directly or indirectly; a “tag” placed on the surface of the delivery device container, usually metallic and biocompatible in nature, which will display to an observer when scanned through external devices such as x-ray, MRI, CT, sound, etc.; and a release mechanism to move the internal contents of the delivery device container into said target cell over a predetermined and specified timed basis, whereby the protective agent internalized in the cell is protecting the cell from subsequent toxicity by a chemotherapeutic agent which is therefore decreasing toxic side effects.

The present invention describes the design, deployment and evaluation of a targeted cytotoxic therapeutic agent in multiple delivery device containers containing Doxorubicin as an agent to kill cells expressing IGF-R. The mAb .alpha.-IR3, selective for IGR-IR, retains full binding and specificity after coupling, and the delivery device container delivers Doxorubicin in its active form. The delivery device container therapeutic agent is more active in vitro and in vivo than free Doxorubicin or free Doxorubicin in combination with mAb .alpha.-IR3. Furthermore, the delivery device containers are highly selective and specific towards cells expressing IGF-R. Moreover, the delivery device container-to-cell transfer mechanism, a syringe, allows bypassing of the p-glycoprotein-mediated resistance both in vitro and in vivo.

The present invention describes the design, deployment and evaluation of a targeted cytotoxic therapeutic agent in multiple delivery device containers containing Taxol as an agent to kill cells expressing p75 receptors. The mAb MC192, selective for p75, retains full binding and specificity after coupling, and the delivery device containers deliver taxol in its active form. The therapeutic agent is more active in vitro and in vivo than free taxol or free taxol in combination with mAb p75. Furthermore, the containers are highly selective and specific towards cells expressing p75.

The present invention describes the design, deployment and evaluation of a targeted neuroprotective therapeutic agent containing a caspase inhibitor as an agent to protect neuronal cells expressing TrkA. The mAb 5C3, selective for TrkA, retains full binding and specificity after coupling, and the delivery device containers deliver the caspase inhibitor in its active form. The therapeutic agent is more active in vitro than free caspase inhibitor. Furthermore, the therapeutic agent is highly selective and specific towards cells expressing TrkA.

The present invention describes the design, deployment and evaluation of a therapeutic agent paclitaxel.cndot.MC192 as an agent to target and kill cells expressing p75 receptors (Guillemard V. et al. Cancer Res. 61:694-699, 2001); the design, deployment and evaluation of a biological “key”, paclitaxel-rabbit anti-mouse antibody as an all-purpose secondary reagent that allows selective tumor targeting with the use of any mouse primary antibody—the paclitaxel-coupled antibodies retain high affinity and specificity, and the delivery device containers deliver the cytotoxic agent in its active form. The therapeutic agent has in vitro cytotoxic activity better than free paclitaxel or free paclitaxel plus free mAb, and also shows high selectivity and specificity towards cells expressing the targeted receptors. In vivo studies show that paclitaxel.cndot.MC192 has a good antitumor activity while free drugs has no effect at equivalent concentrations.

The present invention describes the design, deployment and evaluation of delivery device containers using a therapeutic agent of Doxorubicin with an antibody directed to IGF-R (mAb .alpha.-IR3) for the treatment of IGF-R expressing tumors. Doxorubicin-mAb affords specific and selective toxicity towards cells expressing the targeted receptor. In addition, Doxorubicin-mAb shows better efficacy in vitro than equimolar concentrations of free drug or free drug plus free mAb. Moreover, the container-to-target-cell mechanism, a syringe, bypasses p-glycoprotein-mediated resistance to doxorubicin in tumor cells.

In vivo using a model of human tumors xenografted in nude mice, Doxorubicin as the therapeutic agent in the delivery device container is more efficient at preventing tumor growth and prolonging survival compared to high doses of free doxorubicin or free doxorubicin plus free antibody. Therapy of both the doxorubicin sensitive and resistant tumors is enhanced.

These studies will result in an increase or an improvement of the armamentarium and selectivity of cytotoxic agents. Combinations of other chemotherapeutic agents and other biological “key” molecules using this approach will generate a several fold increase in anti-tumor efficacy.

Another embodiment of the present invention can be provided with protecting agents for specific cells.

The target cell surface marker selected corresponds to the receptor for Nerve Growth Factor: the p140 TrkA tyrosine kinase high affinity receptor. TrkA receptors are expressed on normal cells such as neurons (Saragovi, H. U., and Gehring, K. Trends Pharmacol Sci. 21:93-98, 2000). Monoclonal antibodies have been developed against TrkA, namely mAb 5C3, LeSauteur, L. et al. J. Neurosci. 16:1308-1316, 1996.

The present invention describes the design, deployment and evaluation of delivery device containers using a therapeutic agent of caspase inhibitor peptide (zVAD) with a mAb 5C3 directed to TrkA for the selective protection of apoptotic death in TrkA-expressing neurons. The chemical binding affinity of mAb 5C3 is used to allow the release of the drug inside the target cells. VAD affords specific and selective protection of caspase-mediated apoptosis towards cells expressing the targeted receptor.

In accordance with the present invention, there is provided a method for using a magnetic material as a replacement for the biological “key” molecule and the cell receptor. The opposite poles of the “key” and the receptor would allow the two to bind, which would then be used as the triggering event for the release of the delivery device container's therapeutic agent via the delivery device container-to-target-cell transfer device.

In accordance with the present invention, there is provided a method for using a cell's vibration signature as a replacement for the biological “key” molecule and the cell receptor. The vibration sensor as the “key”, looking for a specific vibration frequency signature, and the cell's own natural vibration as the receptor would allow the two to bind, which would then be used as the triggering event for the release of the delivery device container's therapeutic agent via the delivery device container-to-target-cell transfer device.

Materials and Methods

Synthesis of 2′Glutaryl-Paclitaxel

2′glutaryl-paclitaxel is synthetized by mixing 39 .mu.M paclitaxel (Sigma) with 3 .mu.M glutaric anhydride (Sigma), each dissolved in pyridine, for 3 hours at room temperature. This reaction forms an ester bond at the C2′ position of paclitaxel. The solvent is then removed in vacuo and the residue is dissolved in CHCl.sub.3 and washed with ddH.sub.2O. Purification is achieved by HPLC on a semi-preparative column (Phenomenex); the mobile phase consisted of acetonitrile/water gradient from 35:65 to 75:25 over 50 minutes.

2′glutaryl-paclitaxel (1.334 nmol) is then derivatized with N,N′-carbonyldiimidazole (13.34 nmol) (Sigma) for 25 minutes at 45.degree. C. The carbodiimide reaction activates a carboxylic group on 2′glutaryl-paclitaxel by removing an hydroxyl.

Synthesis of mAb .alpha.-IR3

MAb .alpha.—IR3 (100 .mu.g) is derivatized with 10 mM sodium periodate in 0.1M acetate buffer pH 5.5 for 30 minutes at room temperature. The oxidation reaction is stopped by adding 15 ml of ethylene glycol for 10 minutes at room temperature. This reaction leads to the formation of reactive aldehyde groups.

The by-products are removed by size exclusion and the buffer is exchanged to 1M carbonatelbicarbonate buffer, pH 9.0 using a Centricon device (cut off 50,000 Da).

Cell Lines

The B104 cells are a rat neuroblastoma line that expresses p75 receptors (p75.sup.+). The 4-3.6 cells are B104 cells stably transfected with human TrkA cDNA (p75.sup.+, TrkA.sup.+). NIH 3T3 are mouse fibroblasts that do not express either p75 or TrkA. All cells are cultured in RPMI 1640 supplemented with 5% FBS, L-glutamine, HEPES buffer, and antibiotics.

NIH 3T3 cells are mouse fibroblasts that do not express IGF-R. The NWT-b3 cells are NIH 3T3 cells stably transfected with human IGF-R cDNA. KB cells that are a human nasopharyngeal cancer cell line that overexpress IGF-R and KB-V cells are KB cells resistant to drugs after selection with a constant exposure to Doxorubicin. The mechanism of resistance by KB-V is overexpression of pgp (MDR). All cells were cultured in RPMI 1640 supplemented with 5% FBS, L-glutamine, HEPES buffer, and antibiotics.

Antibodies

MAb MC192 is a mouse IgG1 anti-rat p75 mAb Chandler, C. E. et al. J Biol. Chem. 259:6882-6889, 1984 and mAb 5C3 is a mouse IgG1 anti-human TrkA mAb LeSauteur, L. et al. J. Neurosci. 16:1308-1316, 1996. MC192 and mAb 5C3 are purified and used in culture at 1 nM-5 nM which are near saturating concentrations for cell surface receptors. The “all purpose” secondary rabbit anti-mouse IgG (Sigma) is used in culture at a final concentration of 30 nM. mAb .alpha.-IR3 is a mouse anti-human IGF-R antibody.

Binding Profiles of the Antibodies

FACscan assays are used to measure the receptor binding properties of the antibodies. Cells are immunostained with fluoresceinated-goat-anti-rabbit IgG.

Kinetics of Paclitaxel Cytotoxicity: Single Bolus Versus Constant Exposure

The present invention describes the design, deployment and evaluation of delivery device containers with a therapeutic agent of paclitaxel delivered via the delivery device container approach because affected cells will not synthesize additional target receptors. Therefore, tested is whether a single bolus of paclitaxel is an effective cytotoxic agent. Cells ae exposed to the indicated concentration of paclitaxel for 30 minutes at 4.degree. C. Then, cells are plated in a 96-well plate (Falcon); this group represents treatment with paclitaxel present in a constant manner. The remaining cells are washed free of excess paclitaxel prior to plating; this group represents a single exposure to paclitaxel. The survival profile of the cells is measured using the tetrazolium salt reagent 3-(4,5-dimethylthiazol-2-yl)-2,5-diphenyltetrazolium bromide (MTT, Sigma) 48, 72 and 96 hr after plating. Optical density readings of MTT are done in an EIA Plate Reader model 2550 (Bio-Rad) at 595 nm.

Mechanism of Action of Paclitaxel

B104 cells are plated, 25,000 cells/well in a well plate (Falcon). Free paclitaxel (80 nM) is added to the well and the cells are incubated for 24 hrs. Cells are then treated with Triton 0.01%, 0.1% sodium citrate and 1.mu.g DNAse-free RNAse for 1 hour at 0.degree. C. Nuclei are collected following centrifugation. The DNA is labeled with 75 .mu.l propidium iodide (1 mg/ml stock) in 400 mu.l FACS buffer. All data (3,000 cells/point) is acquired as described above.

In Vitro Cytotoxicity of the Doxorubicin

MTT Assay

For testing the Doxorubicin, cells in 96-well plates (1500-2500 cells/well) are exposed to Doxorubicin, or controls in the presence or absence of Verapamil (6 mu.M). The survival profile of the cells is measured with the MTT assay 72 hr after plating.

Caspase Inhibitor

zVAD=benzoic acid-Valine-Alanine-Aspartic acid-O-methyl-fluoromethyl-Iketone peptide. A known caspase inhibitor, it does not penetrate through the plasma membrane.

In Vitro Survival Assays with Caspase Inhibitor

4-3.6 cells are placed in serum free media (SFM, PFHM-II, GIBCO, Toronto), where they undergo apoptosis (serum-withdrawal model).

Cells are then cultured for .about.48 hrs. Cell growth/survival is calculated relative to 5% serum (standardized to 100%). Addition of serum protects cells in SFM from death. Addition of zVAD does not protect cells because it does not penetrate the plasma membrane.

Cell survival is measured by quantitative tetrazolium salt reagent (MTT, Sigma) and optical density (OD) readings as described [Maliartchouk, 1997]. n=3-6.

In Vivo Tumor Studies for Taxol

Nude mice (seven weeks old, female) are used to test the effect of paclitaxel in tumor progression. Single cell suspensions of B104 cells (10.sup.5/mouse) are injected subcutaneously in the left flank near the rib cage. Tumor growth is monitored daily. Mice are then randomized and treatments are initiated. All treatments are done by a total of five injections every two days (for a total of ten days). All injections are done IP on the right side to prevent direct contact of the agents to the tumor growing subcutaneously. Measurements of tumor volume are taken using calipers, every day post treatment for a total of 25 days.

In Vivo Tumor Studies for Doxorubicin

Nude mice (6-8 weeks old, female) are used to establish relative high and low doses of doxorubicin in tumor progression. Single cell suspensions of NWT-b3 (105/mouse), KB or KB-V (2.5.times. 105/mouse) are injected subcutaneously in the left flank near the rib cage. Tumor growth is monitored daily. After the volume of the tumors had reached 2 mm3, mice are randomized and treatments are initiated. All treatments are done by a total of five injections every 2 days (for a total of 10 days). All injections are done IP on the right side to prevent direct contact of the agents to the tumor growing subcutaneously. Measurements of tumor volume are taken every 2 days post treatment for a total of 20 days.

Statistical Analysis

Statistical significance of differences in tumor growth among the different treatment groups is determined by the student t test using SYSTAT 7.0 software. P value is significant when it is <0.05.

Results

Kinetics of Paclitaxel Cytotoxicity: Single Bolus Versus Constant Presence

Paclitaxel is lipophilic and readily penetrates the cell membrane. In contrast, paclitaxel using cndot antibody “keys” could penetrate the cell via receptor-mediated internalization. The paclitaxel with the cndot.antibody is considered a single bolus because cells affected would cease synthesis of receptor targets. Tested is whether a single short term exposure to paclitaxel can be effective in killing neuroblastoma B104 cells. These assays are done at 4.degree. C. to allow internalization of drug comparable to that afforded by antibody-mediated delivery of paclitaxel via the delivery device container-to-target-cell mechanism approach.

The cytotoxic effect of free paclitaxel is generally the same whether the drug is present in the culture throughout or after a single exposure. Comparable killing is verified at several paclitaxel concentrations. However, a single exposure to 20 nM paclitaxel is significantly less effective than constant exposure after 72 and 96 hours of culture. Likely the amount of drug taken up by the cells after 30 min exposure to 20 nM paclitaxel is sufficient to kill cells over a period of 2 days but not for longer times. These results are encouraging because the cytotoxicity of paclitaxel via delivery device container would be better than that seen after short-term or single exposure to free paclitaxel. Similar data are obtained with 4-3.6 cells.

Binding and Cytotoxicity of Paclitaxel

To assess whether paclitaxel conjugation to antibodies affected antibody binding, this property is tested in FACScan assays (Table 1). Conjugated paclitaxel.cndot.rabbit anti-mouse loses only about.20% of the binding activity compared to unconjugated rabbit-anti-mouse antibody. The binding activity of conjugated paclitaxel.cndot.MC192 stays intact, compared to unconjugated MC192. These results indicate that the method used to conjugate paclitaxel to antibody in a 1:1 ratio does not affect significantly the binding properties of the antibodies. Therefore, there is no difference in binding capability when using the delivery device container with attached antibody provided with this invention versus the conjugate approach of previous inventions.

Binding Fluorescence Experimental Conditions

Testing is done with 4-3.6 cells and B104 cells. Background staining 4.+−.3 3.+−. 0. Paclitaxel-coupled antibody 81.+−. 28 130.+−. 7. Intact antibody 100 4-3.6 cells are bound with mAb 5C3 (10 .mu.g/ml), followed by paclitaxel.cndot.abbit-anti-mouse or intact rabbit-anti-mouse, and goat-anti-rabbit-FITC. B104 cells are bound with paclitaxel.cndot.MC192 (10 .mu.g/ml), or unconjugated MC192 (10 .mu.g/ml), followed by goat-anti-mouse-FITC. Background is assessed by replacing the primary mAb with mouse IgG (10 .mu.g/ml). Cells are analyzed (5,000/assay) by FACScan and LYSIS II software.

The cytotoxic activity of the “all purpose” paclitaxel-anti-mouse conjugate is evaluated in vitro against neuroblastoma cells. The paclitaxel.cndot.rabbit-anti-mouse conjugate is active against cells only when a specific mouse primary antibody is present: mAb 5C3 that binds 4-3.6 cells and mAb MC192 that binds B104 cells. Cytotoxicity is better and more selective than equimolar doses of free paclitaxel. In control assays, conjugate paclitaxel.cndot.rabbit-anti-mouse in the presence of a non-specific primary is not cytotoxic, and the specific primary mAbs in the presence of unconjugated rabbit-anti-mouse are not cytotoxic. Similar analysis using paclitaxel.cndot.MC192 conjugates also shows better activity and selectivity than free paclitaxel at equimolar concentrations. This data suggests the delivery device container with biological “key” antibody attached of this invention will perform at or better than the conjugate approach.

These data also suggest that the paclitaxel.cndot.rabbit anti-mouse conjugate is active by binding to the specific primary antibody, while paclitaxel.cndot.MC192 conjugates are active by directly targeting p75 receptors. The conjugates internalize and release the cytotoxic agent inside the cells. Because only a fraction of paclitaxel.cndot.antibody conjugates can internalize via the targeted receptor, the data suggest that conjugates are significantly much better at cell killing than free paclitaxel, because of both improved transport or penetration. Follow-on testing will determine the increased effectiveness of the container-to-target-cell mechanism approach over the conjugate approach, but the data clearly shows that the present invention exceeds kill rates for free paclitaxel. In addition, due to delivery device container size variations, the present invention can be sized to deliver much higher dosages as well as delivery of these dosages over a predetermined and specified timed basis.

Binding and Cytotoxicity of Doxorubicin

FACScan is used to access whether the .alpha.-IR3 antibody is affected by the chemistry used for conjugation development. The conjugated antibody retains its full binding activity after conjugation compared to free .alpha.-IR3. Results thus indicate that the larger volume and timed release capabilities of therapeutic agent available through the delivery device container approach of this invention will increase the cytotoxicity capability of the drug over the conjugate approach while also removing the unintended systemic side-effects of a free drug regimen.

Testing parameters—binding fluorescence with background staining 10.+−. 1. Doxorubicin-mAb 101.+−. 2. Intact mAb 100 NWT-b3 cells are bound with unconjugated mAb .alpha.IR3 (10 .mu.g/ml), or Doxorubicin-mAb (10 .mu.g/ml mAb equivalent) conjugate, followed by goat-anti-mouse-FITC. Background is assessed by replacing the primary mAb with mouse IgG (10 .mu.g/ml). Cells are analyzed (5,000/assay) by FACScan and LYSIS II software. The data are mean channel fluorescence of bell-shaped histograms, standardized to maximal staining by unconjugated primary antibody .+−. sem. n=5.

The cytotoxicity of the conjugate is initially evaluated in vitro using mouse fibroblasts stably transfected with-IGF-R. KB and KB-V cells are then used to access cytotoxicity as well and to check whether the conjugate could bypass p-glycoprotein-mediated resistance. Cytotoxicity is better than equimolar doses of free Doxorubicin as well as free Doxorubicin plus free antibody while the antibody alone shows no effect on the cells. The conjugate shows unaltered cytotoxicity on KB-V cells which are multidrug resistant as compared to the sensitive KB cells with or without the channel inhibitor Verapamil while free Doxorubicin was inactive on KB-V cells unless Verapamil was added. The results are consistent whether MTT or Colony Formation Assay is done to measure cell death. Results thus indicate that the delivery device container approach of this invention, which allows both higher doses of cytotoxic drugs as well as mixes of drugs in any chemical formula over any time period, will be much more effective at killing tumor cells than the prior art approaches of free drug (systemic) or antigen conjugates.

Test—bypassing of the p-glycoprotein-mediated resistance by the conjugate IC50 (nm).+−. SEM Treatment KB cells, KB-V cells, Doxorubicin 60.+−. 2>320 Dox+mAb 60 .+−. 2>320 Dox+Verapamil 60.+−. 1 150.+−. 1 Dox—mAb 25.+−. 3 30.+−. 4 Dox-mAb+Verapamil 31.+−. 1 30.+−. 2. KB and KB-V cells are treated for 3 days with different concentrations of Doxorubicin, Doxorubicin plus mAb or Doxorubicin-mAb conjugate with or without 6 .mu.M of the p-glycoprotein inhibitor Verapamil. Percent metabolic activity .+−. sem is determined by standardizing untreated cells to 100%. n=3. Representative of 4 independent experiments.

The results indicate that the conjugate can bypass p-glycoprotein-mediated resistance by delivering doxorubicin into the cell by a mechanism not associated with diffusion. The delivery device container of this invention uses a container-to-target-cell mechanism, usually a syringe, to also remove the difficulty of p-glycoprotein-mediated resistance while the delivery device container approach itself allows larger doses, any chemical composition, and timed release regimens.

Selectivity and Specificity of Paclitaxel

The selectivity of paclitaxel.cndot.MC192 conjugate is evaluated using cells that do not express p75. The results show that the conjugate is inactive, while free paclitaxel exhibits dose-dependent cytotoxicity. These results suggest that the activity of paclitaxel.cndot.MC192 conjugates is selective towards cells expressing p75 receptors. The specificity of paclitaxel.cndot.MC192 conjugate is investigated by ligand competition. At 10 nM paclitaxel.cndot. MC192 conjugate (10 nM paclitaxel-equivalent) there is efficient killing of B104 cells. Concomitant addition of 40 nM MC192 blocks cytotoxicity by competing for the p75 receptor target. In contrast, addition of 40 nM non-specific mouse IgG does not affect the activity of paclitaxel.cndot.MC192 conjugates. Cold competition of paclitaxel.cndot.MC192 indicates that death is mediated specifically via p75 receptors. Furthermore, free paclitaxel had the same cytotoxicity whether or not 40 nM of mouse IgG or 40 nM of MC192 antibody are added to the cultures. These results further indicate that the mAbs portion of the present invention stands aside the cytotoxicity issue. The mAbs purpose is purely for target binding. Once completed, this binding process activates the trigger mechanism of the container-to-target-cell mechanism, usually a syringe, which penetrates the cell wall and delivers the therapeutic agent at any dosage and on a predetermined and specified timed basis.

Because some antibodies increase free drug-mediated killing compared to drugs alone, MC192 is tested to determine if it had a pharmacological role as adjuvant. MC192 mAb or mouse IgG did not enhance or decrease the cytotoxicity of various concentrations of free paclitaxel. Similar data were obtained with 60 nM free paclitaxel cultured with increasing doses of antibody. These results indicate that MC192 does not have a pharmacological role, and suggest that MC192 acts only as a carrier and does not contribute to the cytotoxicity of the conjugate in vitro. This data then suggests that the use of a separate binding agent will have no negative effect on the impact of the higher dose of therapeutic agent able to be introduced into the cell via the delivery device container approach presented with this invention.

Selectivity and Specificity of Doxorubicin-.alpha.IR3

The selectivity of the conjugate is evaluated using mouse fibroblasts which do not express IGF-R receptors. The conjugate is totally inactive on those cells, while free Doxorubicin exhibits a dose-dependent cytotoxicity. This result indicates that the conjugate is selective towards cells expressing IGF-R, the targeted receptor. The specificity of the conjugate is evaluated by ligand competition using mouse fibroblasts stably transfected with IGF-R. There is efficient killing by the conjugate which is abolished in the presence of 10 molar excess free .alpha.-IR3. Addition of an excess of mlg does not change the efficiency of the conjugate. Competition of Doxorubicin-.alpha.IR3 indicates that the killing is specifically mediated by IGF-R receptors. Free Doxorubicin has the same cytotoxicity whether excess of mig or .alpha.-IR3 is present or not. The delivery device container approach of this invention removes the negative impact of the IGF-R receptors in the cell killing process. Therapeutic agent(s), in this case Doxorubicin, are injected directly into each cell in amounts larger than can be provided by conjugates, and over any predetermined and specified timed basis, and in cell specific doses without any systemic ill effects.

Cytotoxic Mechanism of Paclitaxel.cndot.MC192 Conjugates

To assess whether the mechanism of action of paclitaxel.cndot.MC192 conjugates is the same as free paclitaxel, cell cycle analysis is done in FACScan assays. The data show that paclitaxel.cndot.MC192 conjugates arrest cells in the G2-M phase of the cell cycle which is consistent with the mechanism of action of free paclitaxel. A G2-M arrest leads to apoptosis in these cells. MC192-treated cells cycle is like untreated control, indicating no effect by the antibody. Again, as above, this circumstance suggests the present invention can use the MC192 antibody as the binding biological “key” element of the system—allowing the therapeutic agent of choice to fulfill all of the killing agent requirements due to the delivery of whatever dosage necessary over whatever predetermined and specified timed basis.

In Vivo Activity of Paclitaxel.cndot.MC192

The antitumor activity of paclitaxel.cndot.MC192 is evaluated in vivo against neuroblastoma xenografted in nude mice. The results show that the conjugate is effective in reducing tumor growth compared to the control (HBSS) (t test, P<0.05), while paclitaxel alone or in combination with MC192 is not able to do so. Moreover, the conjugate prolongs the survival of the mice on average by .about.30% compared to free paclitaxel. This data suggests that a higher dosage of the therapeutic agent—in whatever dosage and chemical combination desired, can be administered with the current invention's delivery device container approach—reducing unintended systemic problems while increasing optimization of the killing process.

In Vivo Efficacy of the Doxorubicin-.alpha.IR3 Conjugate

The antitumor efficacy of the conjugate is evaluated using a mouse fibroblast cell line stably transfected with human IGF-R (NWT-b3) and also in KB cells either sensitive or resistant (KB-V) to doxorubicin, xenografted in nude mice. The results show that the conjugate is more effective at reducing tumor growth, compared to free doxorubicin doses at 50.times higher molar concentration. Survival is also enhanced. This data suggests that the present invention's delivery device container approach can then administer the combined therapeutic agent of Doxorubicin-.alpha.IR3 conjugate but in larger or more targeted doses—expanding and optimizing the chemistry targeting opportunities and the cytotoxicity of the approach without any systemic issues surfacing.

Selective Protection by Selective Inhibition of Caspase

The efficacy of a mAb 5C3-VAD conjugate is tested versus 4-3.6 cells that express the target TrkA (bound by mAb 5C3) and versus B104 cells that do not express TrkA.

Testing 4-3.6 Cell Survival (MTT, % of serum control). Serum free media 0.+−. 4 5%. Serum 100.+−. 2 20 nM zVAD−2.+−. 3 VAD-5C3 conjugate (20 nM VAD equivalent, 1 nM 41.+−. 1 5C3). Neither 20 nM zVAD nor VAD-5c3 conjugate afford protection to B104 cells (parental cells to 4-3.6, but TrkA). MAb 5C3 does not bind to protect B104 cells, and does not protect these cells from death. Typicaly, 0.1 nM mAb 5C3 alone (unconjugated) does protect 4-3.6 cells in SFM to .about.10%, which is a much lower degree of protection than equimolar doses of the VAD-5C3 conjugate. Therefore, the delivery device container approach of this invention can produce the target result of full cell protection by allowing any of various chemical combinations to be placed within the container while also increasing the protective dosage directly injected into the cell over any predetermined and specified timed basis.

2. Discussion

The above provided data shows that mAb MC192 and mAb 5C3, ligands for p75 and TrkA receptors respectively, can be used as carriers for paclitaxel to afford efficient and specific tumor toxicity. An “all purpose” targeting agent can also be developed by paclitaxel conjugation of anti-Ig secondary antibodies.

Kinetics of paclitaxel cytotoxicity. Since the cytotoxicity of paclitaxel.cndot.antibody conjugates is similar to that seen after short-term or single exposure to free paclitaxel, first assessed is whether a single dose of paclitaxel could be efficient at killing the cells. Demonstrated is the fact that paclitaxel cytotoxicity is the same after 48 hours whether a constant or a single dose is given in vitro. This finding underlines the clinical experience of paclitaxel delivery, which is often provided as a single bolus every few weeks, unlike many other chemotherapeutics which are most effective when delivered at low doses over prolonged periods. Our invention allows for both approaches—but increases their effectiveness. The single bolus amount can be enlarged with the simple increase in the size of the delivery device container in order to increase cytotoxicity—or—the invention can provide the dosage of the enlarged container over an extended period—at a predetermined and specified time basis should the latter approach be more effective in killing the target cell.

Improved efficacy. The cytotoxic activity of the conjugates is better than that of free paclitaxel. This is due to better transport, penetration and accumulation of the drug inside the cells—fully supporting the present invention approach of a delivery device container that increases transport efficiency, increases dosage amount, increases chemical compound development without regard to transport or unintended side-effect issues, increases penetration without regard to the p-gp membrane issue, and meters the dosage in a predetermined and specified timed basis.

Improved selectivity. No binding of the conjugates is observed in cells that do not express the target receptors. The paclitaxel.cndot.MC192 conjugate is used for in vivo experiments because it is more suitable than the “all purpose” paclitaxel.cndot.rabbit anti-mouse conjugate. The in vivo experiments confirm the in vitro findings—that the paclitaxel.cndot.MC192 conjugate has a significant antitumor activity against cells expressing p75 receptor in the experimental model used as well as providing a delay in tumor growth compared to other groups. This data underlines the effectiveness of the biological “key” approach of this invention.

The efficacy of the delivery device container and the conjugate compared to free drug is much more evident in vivo than in vitro, due primarily to the in vivo approach providing therapeutic agents in higher concentrations at the tumor site. The effective concentration of conjugate tested in vivo was .about.3.5 nM, whereas free paclitaxel was not effective at this dose. Since the effective concentration of taxanes in humans is in the millimolar range, the therapeutic index of the delivery device container with this invention will rise markedly to the full toxic dosage that can be administered with each container. Furthermore, appropriate in vivo systemic distribution and pharmacokinetics for the delivery device container system of this invention is demonstrated.

Also proven is that there is no obvious toxicity in the treated animals. This is attributed to the fact that both this invention's delivery device container and the paclitaxel.cndot.MC192 conjugate attaches directly and only to the target cell, so it spares non-target expressing cells from any unintended systemic problems.

In review, a general method is proposed to selectively target cancer cells by using the delivery device container for concentrating cytotoxic drugs inside the tumor cells. Furthermore, the present invention provides the capability to couple cytotoxic drugs to small peptidic or non-peptidic ligands of tumor markers—or any other chemistry of choice, as the container protects the body from any of the systemic unintended side-effects. This allows the delivery device container approach to overcome obstacles such as proteolysis, immunogenicity, and poor penetration of solid tumors inherited to antibodies and proteins (Saragovi, H. U., and Gehring, K. Trends Pharmacol Sci. 21:93-98, 2000) when used as therapeutics. The delivery device container also allows any size dose to be specified and delivered directly into the target cell over any predetermined and specified timed basis. The targeted in vivo delivery device container approach of this invention is significantly more effective than 50.times higher molar concentration of free drug—but produces no unintended side-effects.

In vitro, the ligand-caspase inhibitor conjugate demonstrates higher efficacy than free peptide caspase inhibitor because the free peptide can not enter the cell and target the caspases which are located inside the cell. Selective protection of apoptosis is thereby achieved through the more optimal delivery device container approach of this invention.

The present invention produces reduced toxicity and improved therapeutics by selective target-cell-specific delivery of toxic anti-cancer agents. The perfect chemistry and the perfect dosage over the perfect time period now have the capability to be optimized for each cancer type. This allows the dose of chemotherapy to be reduced, non-tumor cells to be largely spared, and resistance to chemotherapy may be reduced.

The present invention also reduces toxicity and improves therapeutics as achieved by this same selective target-cell-specific delivery of protective agents (such as caspase inhibitors, estrogen analogs, or vitamin D analogs) to non-tumor cells such that these cells are spared or protected from death. A person skilled in the art will know that the concept of selective protection can be expanded to degenerative disorders such as Alzheimers disease.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. The whole class, or genus, of variations for a device, method, system and program for intelligent in vivo cell-level chemical or genetic material delivery; wherein multiple injectable biocompatible physical delivery device containers are used to selectively administer medicine, chemical(s) or genetic materials to a target cell in a patient, human or animal, with reduced systemic toxicity; said delivery device container includes an internal contents-to-cell transfer device, an example being a syringe; a biological “key” molecule, magnetic device or vibration frequency signature sensor placed on the surface of the delivery device container which is adapted to selectively bind to said target cell directly or indirectly; a “tag” placed on the surface of the delivery device container, an example being something metallic and biocompatible in nature, which will display to an observer when scanned through external devices such as x-ray, MRI, CT, sound, etc.; and a release mechanism to move the internal contents of the delivery device container into said target cell over a predetermined and specified timed basis.

2. The device, method, system and program of claim 1, for use in targeting a specific cell type or tissue, wherein a digestive enzyme is over expressed in the extra-cellular space of the tissue, and a biological “key” molecule; wherein the biological “key” molecule includes a cell recognition capability that allows the biological “key” molecule to be first attached to the needle opening of the container device and which then, when injected into the body, finds and attaches to the target cells of the target tissue when exposed to the digestive enzyme.

3. The delivery device container of claim 1, wherein said internal contents of which, when a delivery device container becomes bound to said target cell, is internalized into said target cell through the container-to-cell transfer mechanism.

4. The internal therapeutic contents of the delivery device container of claim 1, wherein said therapeutic contents is made up of any member of the group of products known as medicines, chemicals, or genetic materials.

5. The device, method, system and program of claim 1 further comprising: other or additional biological “key” molecules; wherein each biological “key” molecule is directly associated with a recognition segment for a specific target cell or tissue type when said biological “key” molecule is exposed to the digestive enzyme.

6. The biological “key” molecule component of claim 1, wherein said biological “key” molecule is selected from the group of molecules that will bind to a specific target cell via a specific receptor on said target cell. Examples would be the group of antibody and mimicking molecules thereof, monoclonal antibodies, peptides, peptidomimetics, growth factors, hormones, adhesion molecules, viral proteins and functional fragments thereof, etc.

7. The biological “key” molecule of claim 1, wherein when said biological “key” molecule is a primary biologically active molecule indirectly binding to said target cell, said biological “key” molecule further comprises a secondary biologically active molecule selectively bound to the primary and adapted to selectively bind to said target cell.

8. The biological “key” molecule compound of claim 7 wherein said primary and/or said secondary biologically active molecules are an antibody.

9. The biological “key” molecule of claim 8 wherein a primary antibody is of a species and a secondary antibody is of a different species.

10. The “tag” of claim 1, wherein said “tag” is selected from the group of biocompatible markers that allow their position in the body to be displayed when externally scanned by any of the available approaches. The initial embodiment “tag” shall be a gold nano-particle. External scanning device examples would be x-ray, MRI, CT, sound, etc.

11. The device, method, system and program of claim 1, wherein the delivery device container is hydrophilic, biocompatible and/or biodegradable.

12. The contents of the delivery device container of claim 1 being a therapeutic composition, which comprises a therapeutically effective amount of a compound in association with a pharmaceutically acceptable carrier.

13. The contents of the delivery device container of claim 1 being an anti-cancer composition, which comprises a therapeutically effective amount of a compound in association with a pharmaceutically acceptable carrier, wherein said therapeutic agent is a chemotherapeutic agent.

14. A method of claim 1 for treating cancer with reduced effects in a patient, said method consisting in administering a therapeutically effective amount of a compound to a patient, wherein said therapeutic agent is a chemotherapeutic agent.

15. A method of claim 1 for decreasing toxic side effects and increasing selectivity of a chemotherapeutic agent for tumor cells, said method comprising the step of administering to a patient multiple injectable delivery device containers comprising a chemotherapeutic agent, a biological “key” molecule which is adapted to selectively bind to said target cell directly or indirectly, a “tag” particle which is used to display externally the location of the target cell on a scanning or sensing device, and a container-to-cell delivery device wherein said delivery device container therapeutic agent, when said delivery device container is bound to said target cell, is internalized into said target cell.

16. A method of claim 1 for by-passing resistance of tumor cells, said method comprising the step of administering the therapeutic agent of claim 1 to a patient in need of such a treatment whereby said biologically active molecule “key” is a monoclonal antibody and said delivery device container compound is avoiding membrane diffusion and/or permeability route to enter into said cells by the use of a container-to-cell needle mechanism. One such mechanism embodiment shall be a syringe.

17. A method of claim 1 to selectively protect a target cell which comprises a delivery device container compound containing a protective agent to cells selected form the group consisting of: enzyme inhibitors, ligands of nuclear receptors, vitamin D, vitamin E and analogs thereof, estrogen and analogs thereof and inhibitors of the apoptotic case, said method comprising the step of administering to a patient an inject-able delivery device comprising a container with said protective agent, a biological “key” molecule which is adapted to selectively bind to said target cell directly or indirectly, a “tag” particle which is used to display externally the location of the target cell, and a container-to-cell delivery device wherein said delivery device container therapeutic agent, when said container is bound to said target cell, is internalized into said cell on a predetermined and specified time basis.

18. The delivery device container therapeutic agent of claim 1, wherein said therapeutic agent is a protective agent, and further, is an enzyme inhibitor agent.

19. The delivery device container therapeutic agent of claim 18, wherein said enzyme inhibitor agent is a caspase inhibitor agent.

20. The device, method, system and program composition of claim 1, where the composition is injectable through a needle of about 18 gauge or smaller.

21. A method of claim 1 for active embolization in a mammal comprising administering to a mammal in need of treatment multiple biocompatible delivery device containers comprising one or more drugs, vaccines, or combinations thereof.

22. The method of claim 1, wherein the delivery device containers are selected from the group of materials consisting of amorphous metals and alloy mixtures thereof.

23. The method of claim 1, wherein the diameter of the delivery device containers ranges from about 10 nm (nanometers) to about 2000 nm.

24. A method of claim 1 for decreasing toxic side effects and increasing selectivity of non-tumor cells, said method comprising the step of administering to a patient multiple injectable delivery device containers with a protective agent, a “key” molecule which is adapted to selectively bind to said target non-tumor cell directly or indirectly, a “tag” particle which is used to display externally the location of the target cell, and a container-to-cell delivery device wherein said protective therapeutically active drug, when said delivery device container is bound to said target non-tumor cell, is internalized into said non-tumor cell in a predetermined and specified time basis.

25. The method of claim 24, wherein the therapeutically active drug is selected from the group consisting of anti-tumor, anti-angiogenesis, anti-fungal, antiviral, anti-inflammatory drug, anti-bacterial drug, and anti-histamine drug, anti-angiogenic factor, antineoplastic agents, hormones and steroids, vitamins, peptides and peptide analogs, enzymes, anti-allergenic agents, circulatory drugs, anti-tubercular agents, anti-viral agents, anti-anginal agents, anti-protozoan agents, anti-rheumatic agents, narcotics, cardiac glycoside agents, sedatives, local anesthetic agents, general anesthetic agents.

26. The method of claim 25, wherein the vaccine is selected from the group consisting of pneumococcus vaccine, poliomyelitis vaccine, anthrax vaccine, tuberculosis (BCG) vaccine, hepatitis A vaccine, cholera vaccine, meningococcus A, C, Y vaccines, W135 vaccine, plague vaccine, rabies (human diploid) vaccine, yellow fever vaccine, Japanese encephalitis vaccine, typhoid (phenol and heat-killed) vaccine, hepatitis B vaccine, diptheria vaccine, tetanus vaccine, pertussis vaccine, H. influenzae type b vaccine, polio vaccine, measles vaccine, mumps vaccine, rubella vaccine, varicella vaccine, streptococcus pneumoniae Ty (live mutant bacteria) vaccine, Vi (Vi capsular polysaccharide) vaccine, DT (toxoid) vaccine, Td (toxoid) vaccine, aP (inactive bacterial antigen/accelular (DtaP)) vaccine, Hib (bacterial polysaccharide-protein conjugate) vaccine, hepatitis B virus (inactive serum derived viral antigen/recombinant antigen) vaccine, influenza vaccine, rotavirus vaccine, respiratory syncytial virus (RSV) vaccine, human astrovirus vaccine, rotavirus vaccine, human influenza A and B virus vaccine, hepatitis A virus vaccine, live attenuated parainfluenza virus type 3 vaccine, enterovirus vaccines, retrovirus vaccines, and picornavirus vaccines.

27. The method of claim 1, wherein the delivery device container “tags” further comprise a contrast media or a diagnostic agent selected from the group consisting of fluorescent markers derivatives, chemical dyes, and magnetic resonance imaging agents.

28. The method of claim 1, wherein the administration comprises injecting into an area of said mammal in need of embolization.

29. The delivery device containers' therapeutic agent of claim 25, wherein the anti-tumor drug is taxol, doxorubicin, tamoxifen, or a combination thereof.

30. A method of claim 28 for active embolization in a mammal comprising administering to a mammal in need of treatment multiple injectable delivery device containers comprising one or more drugs, vaccines, or combinations thereof, wherein said delivery device containers are delivered to the site of action by the use of targeting antibodies.

31. The device, method, system and program of claim 1, wherein one embodiment has the delivery device container larger than the renal excretion limit.

32. The device, method, system and program of claim 1, wherein one embodiment of the therapeutic drug is a small molecule drug.

33. The device, method, system and program of claim 1, wherein one embodiment of the therapeutic drug is a biomolecular drug.

34. The device, method, system and program of claim 1, wherein one embodiment of the recognition segment is an oligopeptide.

35. The device, method, system and program of claim 1, wherein one embodiment of the recognition segment is an oligosaccharide.

36. The device, method, system and program of claim 1, wherein the target cell or tissue is diseased.

37. The device, method, system and program of claim 1, wherein the target cell or tissue is a tumor.

38. A device, method, system and program of claim 1 containing a pharmaceutical composition comprising a pharmaceutically acceptable excipient.

39. A method of claim 1 of administering a drug to a patient, the method comprising steps of: providing a patient; providing a pharmaceutical composition that comprises a pharmaceutically acceptable excipient and an effective amount of the therapeutic agent in multiple delivery device containers of claim 1; and administering the pharmaceutical composition to the patient.

40. A method of claim 1 for using a magnetic material as a replacement for the biological “key” molecule and the cell receptor. The opposite poles of the “key” and the receptor would allow the two to bind, which would then be used as the triggering event for the release of the delivery device container's therapeutic agent via the delivery device container-to-target-cell transfer device.

41. A method of claim 1 for using a cell's vibration signature as a replacement for the biological “key” molecule and the cell receptor. The vibration sensor as the “key”, looking for a specific vibration frequency signature, and the cell's own natural vibration as the receptor would allow the two to bind, which would then be used as the triggering event for the release of the delivery device container's therapeutic agent via the delivery device container-to-target-cell transfer device.

42. The device, method, system and program composition of claim 1, where the device, method, system and program for administering the therapeutic agent or the delivery device containers to the patient is a transdermal patch rather than a standard injection, wherein the micro-needles of the transdermal patch are made of amorphous metal alloys.
